(19) United States
(12) Patent Application Publication
(10) Pub. No.: US 2004/0033623 A1
(43) Pub. Date: Feb. 19, 2004

(54) Assay for detection of antigen in bodily fluid

(75) Inventors: Jyotsna Shah, Nashua, NH (US); Nick Harris, Monterey, CA (US); Hua Bai, Palo Alto, CA (US); Georgiana Remollo, San Jose, CA (US)

Correspondence Address: Kevin M. Farrell, Pierce Atwood, One New Hampshire Avenue, Suite 350, Portsmouth, NH, 03801, US

(73) Assignee: IGeneX, Inc., Palo Alto, CA

(21) Appl. No.: 10/222,947

(22) Filed: Aug. 16, 2002

(51) Int. Cl. G01N 33/554; G01N 33/569; G01N 33/543

(52) U.S. Cl. 436/518

(57) ABSTRACT

Disclosed is a method for detecting a target antigen in a fluid sample of an individual. In preferred embodiments, the fluid sample is urine, cerebral spinal fluid, or synovial fluid. After an optional concentration step, the fluid sample is passed through a membrane suitable for binding the target antigen, thereby binding the target antigen to the membrane. Target antigen, bound to the membrane, is then detected using an antibody specific to the target antigen.

### BACKGROUND OF THE INVENTION

Over the years a number of methods have been developed for diagnosing disease caused by pathogens. Traditionally, such diagnosis has relied on serological findings and/or clinical evaluation of patients. Serological techniques are invasive, requiring obtaining blood samples from patients and processing of samples within a relatively short period of time. Moreover, because serology is often based on detection of antibodies to the pathogen, the technique has been of limited use since there may be no immune response to a pathogen for some time. Even if the presence of antibodies in a patient's blood is determined, such a finding does not always prove active disease.

Diagnosis based on clinical assessments, while non-invasive, have also been of limited use because they are often based on presentation of symptoms. Diagnosis made according to the presence of symptoms can be subjective, from both the clinician's perspective, as well as the patient's. As such, the likelihood of misdiagnosis based on clinical assessments has been problematic.

In more recent years research has been aimed at development of reliable and non-invasive biochemical and immunological methods that can detect pathogens in body fluids. One such technique involves the use of polymerase chain reaction (PCR). According to PCR, oligonucleotide primers that specifically hybridize to genomic DNA sequences of a particular pathogen are used to amplify a segment of the genomic DNA of the pathogen whereby detection of an amplification product is indicative of the presence of the pathogen in the fluid specimen. Although PCR can be a highly sensitive technique, in the absence of very extensive sequence information, it may lack sufficient specificity since diverse pathogens may have highly homologous regions of DNA. Also, from a cost perspective, PCR techniques can be very expensive, requiring special equipments such as thermocyclers and the routine maintenance of designated and isolated areas that are free of nucleic acid contamination.

Methods have also been developed for the detection of pathogens present in body fluids that are based on immunology. Immunochemical methods detect pathogens using antibodies that specifically recognize antigens expressed by the pathogen. The antigen that is recognized may either be present inside the pathogen (i.e. intracellular), or it may be displayed on the surface of the pathogen, or shed in the body fluid. Nevertheless, according to immunochemical methods, antigen-specific antibodies bind to antigens present in the fluid specimen, and the antigen is detected using any number of commonly available detection reagents. Broadly, there are two types of immunochemical techniques, namely, immunoblotting and immunoassays.

Immunoblotting combines gel electrophoresis with immunochemical detection. In brief, immunoblotting involves preparation of the antigen sample, resolution of the sample by gel electrophoresis, transfer of the electrophoresed sample to a membrane support, and detection. Although highly reliable and sensitive, immunoblotting is tedious and time consuming as it requires additional and nontrivial steps, including electrophoresis of the sample prior to antigen detection. Testing or screening for antigens using immunoblotting can be inefficient and expensive, and is not well-suited for high throughput adaptations.

A common alternative to immunoblotting are immunoassays, such as enzyme-linked immunosorbent assay (ELISA). Although versatile, immunoassays can be inaccurate and lack sensitivity. Immunoassays are also tedious and require special equipment such as microtiter plates, special readers, and special aspirators for washing. Also the actual signals generated cannot be archived (i.e. only the plate reader numerical print-outs can be stored). In addition, a major problem that is inherent to immunoassays is the substantial interference by blood cells that may be present in the body fluid with the assay.

Therefore, in light of inherent limitations of the prior art methods for detecting antigens specific to pathogens in bodily fluids, it would be desirable to provide a new and simple method for detecting such antigens that is sensitive and specific, and gives reproducible results. It is desirable that the method be sufficiently versatile so that it could be used in a variety of settings, including clinics and laboratories, and, if desired, be performed non-invasively. Moreover, it is desirable that the method be easy to use and cost-effective, and require no special equipment.

### SUMMARY OF THE INVENTION

The present invention relates to a method for detecting a target antigen in a fluid sample of an individual. In preferred embodiments, the fluid sample is urine, cerebral spinal fluid, or synovial fluid. After an optional concentration step, the fluid sample is passed through a membrane suitable for binding the target antigen, thereby binding the target antigen to the membrane. Target antigen, bound to the membrane, is then detected using an antibody specific to the target antigen.

### DETAILED DESCRIPTION OF THE INVENTION

The present invention relates to an improved and simplified method for detecting the presence of a target antigen in a fluid sample obtained from an individual. An important aspect of the invention is that the fluid sample obtained from the individual, which is to be tested for the presence of an antigen, is processed in a particularly simple and convenient manner that is well-suited to application in a clinical laboratory. More specifically, the fluid sample (or a concentrated form thereof) is simply passed through a membrane which will bind antigen based on charge. The unexpected finding was that antigens that were bound to the membrane in this simplified manner could subsequently be detected with great sensitivity, specificity, and reproducibility using antibodies specific to the antigen. In fact, as demonstrated in the Exemplification section which follows, the assay was far superior than an ELISA-based assay for the detection of antigen of the spirochete responsible for Lyme disease. The method of the present invention was superior in terms of specificity, sensitivity, and reproducibility.

As previously mentioned, the present invention represents an advance in the art not merely from a performance standpoint, but also from a convenience standpoint. The ability to simply pass a fluid sample through a filter (after optionally concentrating the sample) requires only the most rudimentary equipment and minimal training. As will be discussed in greater detail, not all bodily fluids are suitable for use in connection with the methods of the present invention.

Antigen, as that term is used herein, is an epitope-containing protein or polypeptide. Generally speaking, such epitope-containing molecules are comprised of a minimum of 6-8 amino acid residues. Such epitope-containing molecules can, of course, be the subject of post-translational modification and may include, therefore, glycoproteins or lipoproteins. Antigens to which the present invention are directed are antigens that are specifically expressed in association with a pathological condition. An additional requirement is that the antigen must be present in detectable quantites (with optional concentration) in one of the bodily fluids for which the method of the present invention is applicable. It will be recognized by those of skill in the art that any antigen, present in one of the specified bodily fluids in sufficient concentration, can be detected using the method of the present invention.

It is known in the art that antigen associated with a particular pathological condition may be preferentially distributed in one bodily fluid relative to another. This factor must be taken into account when carrying out the methods of the present invention. For example, an antigen such as a secreted toxin may be shed into a particular bodily fluid by a cell which expresses the toxin. Alternatively, the antigen may be attached to the surface of the pathogen present in the fluid sample or it may be expressed intracellulary. The antigen can also be, for example, expressed by a virus that has infected a cell of an individual or has been shed into the fluid of the individual.

Those skilled in the art will recognize that prior art publications provide a great deal of information as to the type of bodily fluid sample that is best suited for use for detecting a specific antigen. Where such information has not been documented in the literature, it is a routine matter to determine the information. For example, one skilled in the art can, without undue experimentation, analyze by traditional methods, such as PCR or western blot, which type of fluid from an individual known to be infected by a specific pathogen is best suited for analysis of the type described herein.

In a preferred embodiment, the method of the present invention is performed using a bodily fluid which is obtained non-invasively. An example of a fluid sample which can be obtained non-invasively is urine. In instances in which the use of urine is not suitable for detection of a particular antigen, it may be necessary to resort to invasive techniques for obtaining the bodily fluid sample. Other types of bodily fluid samples that can be analyzed by the methods disclosed herein include cerebral spinal fluid (CSF) and synovial fluid. Methods for obtaining such samples are well-known in the art.

As discussed, the type of bodily fluid selected for analysis will depend to some extent on the likelihood that the antigen to be detected will be present in that fluid in a form, and at a concentration, sufficient for detection (with optional concentration). For example, it is documented in the literature that Borrelia burgdorferi, the causative agent of Lyme disease, is found in the bladder, and its antigens are shed in the urine. Thus, for example, patients suspected of Lyme disease can be non-invasively screened for infection by B. burgdorferi by analysis of their urine. In fact, in Example 1 of the Exemplification section that follows, the application of the present invention to the detection of B. burgdorferi antigens is demonstrated.

If the fluid sample to be analyzed contains gross macroscopic debris, for example as determined by visual inspection, the fluid sample can be cleared of such debris by an optional low speed centrifugation step appropriate for sedimenting the debris, but not the antigen in the fluid sample. For example, the specimen can be cleared by low speed micro-centrifugation at about 1000 rpm for 5 min, whereby the debris-containing pellet is discarded and the supernatant from this step, which contains the target antigen, is analyzed.

In one embodiment, the antigen in the fluid sample is optionally concentrated prior to passing of the fluid sample through the membrane. Such an optional step may be performed where the concentration of the antigen may be low and the amount of volume of fluid sample that can pass through the membrane is limited. Thus, although any volume of fluid sample can be passed through the membrane, it may be desired to limit the volume, and this can be achieved by the optional concentration step. Concentrating the fluid sample can also make possible the collection and pooling of multiple fluid samples obtained at different times from the same individual. Concentration of fluid samples can also be practical in that it allows for sample volume adjustments, especially where a predetermined sample volume is desired, for example in automated high through-put processing.

One skilled in the art will recognize that concentrating antigen in a fluid sample can be accomplished by a variety of routine techniques common in the art. In some cases, the choice of technique will depend on the size of the antigen. In the preferred embodiment, the target antigen is concentrated by, for example, micro-centrifugation at about 13,000 rpm for 10 min. In some cases, ultra-centrifugation will be required to pellet the antigen.

Alternatively, the antigen can be concentrated by allowing, or promoting, evaporation of liquid from the fluid sample. For example, evaporation of liquid from the sample can be achieved by using an apparatus known in the art as a “speed vac”, wherein fluid samples in open containers are spun in a centrifuge with heat and under vacuum, thereby promoting evaporation of the liquid. Evaporation of liquid will decrease the volume of a fluid thereby increasing the concentration, or titer, of the antigen in the fluid sample.

The antigen can also be concentrated by precipitation of antigen followed by resuspension in a suitable volume of a solution. Precipitation techniques are known in the art, and are a matter of routine procedure.

Prior to passing the fluid sample through a membrane, and if necessary, the antigen present in the fluid sample can be denatured using an appropriate denaturing solution. Those skilled in the art recognize that some antigens may require denaturation, for example with detergent and boiling, in order to permit binding of antibody to the antigen. This can occur, for example, when the epitope recognized by the antibody is buried within the tertiary structure of the epitope-containing molecule and inaccessible to the detecting antibody. In such cases, antigen can be pelleted and resuspended in a denaturing solution. Alternatively, reagents capable of denaturing the antigen (e.g., the denaturing detergent Triton) can be added to the fluid sample. Other techniques for denaturing a molecule in a solution are known in the art and can be employed.

Following the initial preparatory steps described above, the fluid sample is passed through a membrane that is suitable for binding the target antigen present in the fluid. Suitable membranes used to bind proteins are known in the art and include, for example, nitrocellulose, activated paper, and activated nylon. In particular, charged membranes are suitable for binding epitope-containing molecules such as protein or polypeptides.

A simple gravity-flow filtration apparatus can be suitable for passing the fluid through a membrane. Typically, in such a situation, the fluid will be loaded in the apparatus late in the day and allowed to flow through the membrane overnight. Alternatively, the sample is passed through the membrane under vacuum using, for example, a micro-filtration device such as the “Bio Dot” apparatus (Bio-Rad). Centrifugation can also be used to speed up the passage of the fluid sample through the membrane. Those skilled in the art recognize that passing the fluid sample through the membrane can be accomplished in a variety of other ways not specifically mentioned here.

In the preferred embodiment, the target antigen in the fluid sample is an antigen that is expressed by a pathogen that has infected an individual. Detection of the antigen will be indicative of the presence of the pathogen in the fluid of the individual. For example, the pathogen can be a bacteria, a parasite, a fungus, a mold, or a virus. Preferably, and as described in the Exemplification section which follows, the pathogen to be detected is B. burgdorferi, a known causative agent of Lyme disease. The most dangerous time for infection with B. burgdorferi is from April to September, a period when people are more apt to be outdoors where they may encounter the B. burgdorferi-infested ticks. Often a characteristic “bulls eye” rash appears at the site of the tick bite, sometimes followed by a flu-like illness. There may be no apparent immune response for several weeks to several years, depending on B cell antibody production. B cell activity may be detected as an IgM response in two to four weeks, or as an IgG response in four to six weeks. However, the presence of antibody does not always prove active Lyme disease. It is documented in the literature that B. burgdorferi, the causative agent of Lyme disease, is found in the bladder, and its antigens are shed in the urine.

In order to test and validate the instant invention, urine and CSF samples were analyzed for the presence of B. burgdorferi antigen by the method disclosed in the present Application. To detect B. burgdorferi, antibodies to B. burgdorferi antigens 18 kD, 23-25 kD, 30 kD, 31 kD, 34 kD, 39 kD, 45 kD, 58 kD, 66 kD, and 83/93 kD can be used as target-specific antibodies. Membranes can be incubated with the target-specific antibody under conditions appropriate for binding of the target-specific antibodies to the target antigens. Depending on the specific antigen and/or antibody employed, incubation parameters may require routine optimization. It should be noted that immunochemical detection protocols are well known in the art and optimized conditions for antigen detection can be determined with minor adjustments and undue experimentation.

In the preferred embodiment, the target antigen can be detected using an “indirect” detection assay. For indirect detection, a labeled secondary reagent that will bind specifically to the target-specific antibody is used. The secondary reagent can be an antibody (i.e. secondary antibody) and the label that is attached to the antibody can be, for example, enzymes, radioactive isotopes, biotin, or fluorochrome. The choice of label will depend on the type of detection method and signal desired. In the preferred embodiment, a chromogenic signal is preferred. For example, for the detection of Lyme antigens, the secondary reagent is goat anti-rabbit antibody labeled with horseradish peroxidase (HRP). Following binding of the primary antibody to the antigen, the HRP-labeled secondary antibody is added which binds to the primary antibody. The resultant complex, comprising antigen, primary antibody, and HRP-labeled secondary antibody, is then incubated with the chromogenic substrate, tetramethylbenzidine (TMB). The presence of the antigen is indicated by the appearance of a blue color spot on the membrane where the antigen is bound. Other variations of “indirect” detection assays are known in the art and can be used with the instant invention.

In another embodiment, the target antigen can be detected using a “direct” detection assay where the antigen-specific antibody is labeled with a reporter moiety and a secondary antibody is not employed. For example, the antigen-specific antibody, the primary antibody, can be labeled similar to the labeling used with the “indirect” detection method described above. Thus, for example, the antigen-specific antibody can be labeled with HRP. In another variation, biotin-coupled antigen-specific antibodies can be detected with HRP-labeled streptavidin. Variations of “direct” detection assay for detecting antigen are well known in the art and can be used with the present invention. Other suitable secondary reagents can be used depending on the type of target-specific antibody employed and the detection method desired, although chromogenic detection methods are preferred.

The signal that is generated from the reporter moiety or label will be an indicator of the presence of the target antigen in the fluid sample. As described above, in the preferred embodiment, where the reporter label is HRP, a chromogenic TMB membrane substrate is allowed to react with HRP to generate a blue color. The signal, which is in the form of a blue precipitate (color reaction will produce a blue color dot), will appear at the reaction site in the presence of HRP. Importantly, this result can be stored a number of ways. For example, the membranes can be stored in a cool place for a relatively long period of time without loss of signal. Alternatively, the membranes can be scanned into a computer as a picture, the intensity of the signal adjusted, as needed, according to control and background levels, and the data stored in a computer file. The intensity of the signal can be proportional to the concentration of the antigen in the sample. A sample with a signal intensity equal to or greater than the limit of detection, as determined by parallel processing of appropriate positive and negative controls, can be reported as positive.

### EXEMPLIFICATION

### EXAMPLE 1

Detection of Lyme Antigen in Urine

Lyme Dot Blot Assay (LDA) is Specific, Sensitive, and Reproducible

The method of the present invention, for detecting Lyme antigen in fluid samples, is referred to as the Lyme Dot Blot Assay (LDA). To determine the sensitivity, specificity, and reproducibility of LDA, urine samples with or without Lyme antigen were tested for presence of sonicated B. burgdorferi. For comparison of the LDA method to another commonly used assay for detection of Lyme antigen, parallel urine samples were also analyzed using the Lyme Urine Antigen Test (LUAT), a method that utilizes an enzyme-linked immunosorbant assay (ELISA) format.

Twenty urine samples each were tested for Lyme antigen by LDA and compared to LUAT. An identical set was also tested again on a different day (designated as “day 2”) in order to determine reproducibility of the methods. Each set included 6 urine samples spiked with a range of concentration of sonicated B. burgdorferi (isolates B31 and 297) and 14 Lyme negative samples. Detection values equal to or greater than 32 ng/ml were interpreted as positive results. The results are shown in Table 1.

In the first run (day 1), the LDA method detected Lyme antigen in all 6 urine samples spiked with Lyme antigen, but not in the negative non-spiked samples. Moreover, the sensitivity of detection by LDA was in the range of 25-400 ng/ml of Lyme antigen. In contrast, the LUAT method detected only four of the six Lyme antigen-spiked samples. The concentration of Lyme antigen in these four samples ranged between 100-400 ng/ml. Therefore, according to the results on day 1, LDA was 100% sensitive and 100% specific. Although LUAT was also 100% specific, the method was only 66.7% sensitive.

When the test was repeated on day 2, LDA continued to be 100% sensitive and 100% specific (see Table 1, LDA, day 2). In contrast, LUAT detected only 3 of the six positive samples (50% sensitivity) and reported 2 of the 14 negative samples as positive (two false positive=86% specificity). In contrast to LDA, LUAT was not reproducible as demonstrated by the differing results obtained on days 1 and 2. Taken together, the results demonstrate that the LDA method is sensitive, specific, and reproducible.

Further studies have shown that LDA has a specificity of at least 95% and a sensitivity of about 12.5 ng/ml with respect to detection of B. burgdorferi in urine. LDA performed in conjunction with a PCR-based method (e.g. Lyme Multiplex PCR) increases the sensitivity of detection of B. burgdorferi in urine by about 50%. In addition, a reverse western blot (RWB) assay for detection of B. burgdorferi antigens in urine is useful for confirmation of LDA positive urine samples.

Finally, the Statistics Department at UC Davis, CA statistically analyzed data from over 500 LDA positive patients, submitting 3 urine samples collected on 3 different days. The study results indicated that the chance of finding a positive by LDA is approximately 65% with 2 urine samples and 45% with 1 sample, albeit with 3 urine samples the chance of finding a positive is 100%.

Interference Study

To ascertain whether specific chemical or cellular factors, when present in a specimen, interfere with LDA results, urine samples containing potentially interfering substances, such as leukocytes, red blood cells, and/or nitrites, were analyzed by Dipstix-7 for the presence of interfering substance prior to being tested by LDA. To confirm whether a sample was negative or positive for Lyme antigen, samples were also tested by PCR or RWB methods. Any sample that was negative by LDA and RWB, or LDA and PCR was considered a “true negative”. Any sample that was positive by LDA and RWB, or LDA and PCR was considered a “true positive”. The results are shown in Table 2 and summarized below:

i) Leukocytes Do Not Interfere with LDA

All urine samples which were confirmed as negative for Lyme antigen by the RWB method, and that contained 1+, 2+, or 3+ leukocytes, were determined to be negative for the Lyme antigen by the LDA method, suggesting that the presence of leukocytes in a Lyme antigen-negative sample will not yield a “false positive” result (see sample #1-11). Moreover, the results of samples 13, 15, and 16 suggest that the presence of leukocytes in a Lyme antigen-positive urine sample does not interfere with LDA to yield “false negative” results.

ii) Nitrites May Interfere with LDA

Of the 9 specimens that contained nitrites, 5 samples were determined to be positive for the Lyme antigen by LDA and confirmed as true positive by RWB (see samples 13, 15, 16, 17, and 20). However, 4 of the true negative samples that contained nitrites tested positive for Lyme antigen according to LDA (see samples 12, 14, 18, and 19). These results suggest that nitrites, if present in a specimen, may interfere with LDA. Therefore, any urine sample that tests positive for nitrites by the Dipstick-7 will be rejected or if tested, and is positive, must be confirmed by either PCR or RWB.

iii) Red Blood Cells Can Interfere with LDA

Nine urine samples that contained 3+ red blood cell levels, as measured by Dipstick-7, were analyzed by LDA. Of these samples, two were positive for Lyme antigen according to LDA results. However, when these same two samples were tested for Lyme antigen by the RWB method, they were determined to be negative (see samples 27 and 28). This suggests that these two samples were “false positive.” Similar to samples that contain nitrite, samples containing 3+ red blood cells according to the Dipstick-7 will be rejected or if tested, and are positive, must be confirmed by either PCR or RWB.

### EXAMPLE 2

Detection of Lyme Antigen in Cerebral Spinal Fluid

The protocol for detection of B. burgdorferi in CSF is essentially similar to the protocol used for urine. Because CSF samples were significantly less than urine samples, CSF was not concentrated by the optional concentration step. Under these circumstances, a 100 μl aliquot of CSF was directly processed by addition of 10 μl of 0.01% Triton in the same way as the urine samples. CSF samples, obtained from non-Lyme patients, were either spiked with varying concentrations of Lyme antigen, as described before, or with whole blood. Spiked samples were analyzed by LDA and the results compared to non-spiked control CSF samples. The results are shown in Table 3 and summarized below.

All control CSF samples were negative (see samples 1-10) and all CSF samples that were spiked with Lyme antigen were positive (see samples 11-22), showing that the assay is reliable also with respect to detection of Lyme antigen in CSF. Interestingly, LDA can detect as low as 15 ng/ml of Lyme antigen in CSF (see samples 20-22). There was no interference from whole blood in that all samples spiked with whole blood were negative (see samples 23-34). Based on these results, CSF LDA has high specificity and sensitivity.

Methods of the Invention

Materials

Protran, 0.2 μm pore size nitrocellulose membrane was obtained from Schleicher & Schuell (Catalog #5077-04). Sonicated B. burgdorferi antigen in TBS Buffer (isolate #B31 and 297) were obtained from Dr. Denee Thomas of University of Texas at San Antonio. Primary antibody polyclonal rabbit anti-B. burgdorferi antibody in Tris buffer (pH 7.4) was obtained from Strategic Biosolutions. Secondary antibody horseradish peroxidase conjugated goat anti-rabbit antibody (Catalog #474-1516) and TMB membrane substrate (Catalog #5077-04) were obtained from KPL. Dry blend Superblock was purchased from Pierce (Catalog #3745). 10× Tris Buffered Saline (Catalog #1706435) and Tween 20 (Catalog #170-6531) were purchased from Bio-Rad. Bovine Serum Albumin, Grade V (Catalog #BP 1600-100) was obtained from Fisher, and Triton X-100 (Catalog #T-9284) was from Sigma.

Reagents

1× TBST, pH 7.5

900 ml dH₂O and 100 ml 10× TBS and 0.5 ml Tween-20 (0.05% Tween)

1× Blocking Solution

200 ml dH₂O and 1 pouch dry blend Superblock

Primary Antibody Diluent (1% BSA/TBS)

0.5 g BSA and 50 ml 1× TBS

Working Solution of Primary Antibody (20 ml Per Blot)

(prepare just before use) 20 ml 1% BSA/TBS and an amount of polyclonal rabbit anti-B. burgdorferi antibody (e.g., anti-18 kD, anti-23-25 kD, anti30 kD, anti-31 kD, anti-34 kD, anti-39 kD, anti-45 kD, anti-58 kD, anti-66 kD, and anti-83/93 kD) sufficient for detecting B. burgdorferi antigen.

Secondary Antibody Diluent (1% BSA/TBST)

0.5 g BSA and 50 ml TBST

Working Solution of Secondary Antibody (20 ml Per Blot)

(prepare just before use) 20 ml 1% BSA/TBST and 1.5 μl peroxidase-conjugated, affinity purified goat anti-rabbit IgG (H+L) with minimal cross-reaction to human serum proteins (Catalog #474-1516, KPL)

Lyme Antigen Stock

Sonicated B. burgdorferi isolates B31 and 297 (1:1) at 1 mg/ml in TBS buffer.

Fluid Samples Procurement

Urine samples can be in the form of a preserved or an unpreserved specimen. Unpreserved urine samples can be clean-catch and mid-stream urine samples that have been freshly voided in a clean container. If unpreserved specimens are not to be analyzed immediately, they can be stored frozen at a temperature in the range of about −10 to −20° C. for about 2 months.

Urine sample can also be collected as a preserved specimen using, for example, a B/D Urine Vacutainer kit (B/D catalog #4962). Preserved urine samples are stable for about 7 days at room temperature. In the case where Lyme antigen is to be detected in a urine specimen, it is preferred that first morning urine be used since it typically contains the highest concentration of Lyme antigen.

CSF samples were obtained from an individual by standard protocols known in the art. In brief, CSF is obtained by inserting a needle with stylet at the interspace between L3-L4, L4-L5, or L5-S1 vertebrae. Upon reaching the subarachnoid space, the stylet is removed and 2 to 5 ml of fluid is collected into a leak-proof sterile screw cap tube. The tube can be sent at room temperature to the laboratory, if the sample is to be analyzed immediately. If samples are not shipped immediately, they can be frozen and delivered to the laboratory on dry ice for storage and subsequent analysis at a later date.

Immobilization of Pathogen Antigen on Membrane

Patient urine specimens and control samples were briefly vortexed and centrifuged at 1000 rpm for 5 min (Baxter Biofuge 15). This low centrifugation spin is optional and is intended to clear the sample of macroscopic debris. The supernatant (1.4 ml) was transferred to clean tubes and spun at 13,000 rpm for 10 min to pellet the antigen to be detected. The supernatant (˜1.3 ml) was discarded and the pellet was resuspended using the remaining ˜100 μl of supernatant. Following the addition of 10 μl of 0.01% Triton, samples were incubated at room temperature for about 30 min, followed by boiling for about 5 min, to allow solubilization of the samples. To immobilize the antigen on a membrane in preparation for detection with antigen-specific antibody, samples were passively filtered overnight onto a prewetted nitrocellulose membrane using a vacuum micro-filtration apparatus (Bio-Dot by Bio-Rad).

Patient CSF specimens were directly solubilized without performing the optional concentration step. A 100 μl aliquot of CSF was processed by direct addition of 10 μl of 0.01% Triton. All subsequent steps were performed essentially as described for urine samples.

Detection of Pathogen Antigen with Antibody

After overnight filtration of the solubilized pellet, the nitrocellulose membranes containing the bound antigen were washed with 1× TBST (three times; 5 min each time) and dried on blotting paper for about 10 min. Membranes were then blocked in 1× Superblock (15 ml/membrane) at room temperature for about 40 min with gentle rocking on a horizontal rocker at 10-12 rpm. Following a 1× TBST rinse, membranes were then incubated with a working solution (see Materials) of primary rabbit anti-B. burgdorferi antibody for one hour at 37° C. with gentle rocking. Membranes were then washed 3 times in 1× TBST, incubated with 20 ml of a working solution (see Materials) of a horseradish peroxidase conjugated secondary antibody for 1 hr at room temperature, and washed with 1× TBST. The chromogenic TMB membrane substrate was allowed to react with the HRP moiety for 20 min with gentle rocking before membranes were rinsed with deionized water and dried overnight on blotting paper in a dark place.

Controls and Calibrators

High positive control (50 ng/ml Lyme antigen), weak positive control (12.5 ng/ml Lyme antigen), negative control, and spiked calibrators (0, 3, 6, 12.5, 25, and 50 ng/ml Lyme antigen/ml) were included in every run to confirm reproducibility, sensitivity, and specificity of the test procedure in the same format as the patient samples. The controls were expected to have the following coloration pattern:

i) negative control=no coloration

ii) 3 and 6 ng/ml controls=no to very weak blue coloration.

iii) 12.5, 25, and 50 ng/ml controls=weak to high blue coloration.

The testing was considered satisfactory only if the high positive and weak positive controls had blue positive coloration and the negative control had no visible blue coloration.

Interpretation and Reporting Results of LDA

A blue precipitate, in the form of a blue dot on the membrane, will appear at the reaction site between HRP and TMB, and its intensity will be proportional to the concentration of the antigen in the sample. Membranes that were treated with TMB and dried overnight, were scanned into the computer (HP Scanner 6300C). Background levels were adjusted, consistent with negative and positive controls as described above.

Samples having a blue color intensity equivalent to or greater than the color intensity of the 12.5 ng/ml calibrator sample were considered “presumptive positive.” Presumptive positive samples were subject to confirmation by another method, such as PCR or reverse western blot (RWB). Patient samples with color intensities less than that of the 12.5 ng/ml calibrator were considered negative, a result that does not exclude a diagnosis of the disease caused by the pathogen. Finally, specimens whose intensities were ambiguously positive (blue color) were repeated.

Lyme Urine Antigen Test (LUAT)

In brief, Lyme antigen in the specimen, calibrators, and controls were mixed with an antigen-specific antibody conjugated to a reporter enzyme to form Lyme antigen/antibody complexes. The antigen/antibody complexes were added to microtiter wells previously coated with Lyme antigens. In this way, the “free” Lyme antigens in the samples essentially compete with the immobilized antigens in the wells for limited binding sites in a homogenous reporter enzyme conjugated polyclonal antibody solution. After washing, fluorogenic substrate was added and allowed to react with the reporter enzyme component of any antibody that remained bound to the immobilized antigens in the wells. The generated fluorescence was measured in an FF96 fluorometer and reported in units called FSU. The intensity of fluorescence is indirectly proportional to the concentration of the antigen being measured, wherein high antigen concentration in the sample results in low FSU signal. In repeated experiments, the sensitivity of the LUAT was determined to be about 32 ng/ml (Lyme antigen/ml).

TABLE 1

| | LUAT | LUAT | LDA | LDA | |
|---|---|---|---|---|---|
| Sample # | Day 1 | Day 2 | Day 1 | Day 2 | Expected |
| 1 | Negative | Negative | Negative | Negative | Negative |
| 2 | Negative | Negative | Negative | Negative | Negative |
| 3 | Negative | Negative | Negative | Negative | Negative |
| 4 | Positive | Positive | Positive | Positive | Positive |
| | | | | | (400 ng/ml) |
| 5 | Negative | Negative | Negative | Negative | Negative |
| 6 | Negative | Positive | Negative | Negative | Negative |
| 7 | Negative | Positive | Negative | Negative | Negative |
| 8 | Positive | Positive | Positive | Positive | Positive |
| | | | | | (200 ng/ml) |
| 9 | Negative | Negative | Negative | Negative | Negative |
| 10 | Negative | Negative | Negative | Negative | Negative |
| 11 | Positive | Negative | Positive | Positive | Positive |
| | | | | | (50 ng/ml) |
| 12 | Negative | Negative | Negative | Negative | Negative |
| 13 | Negative | Negative | Negative | Negative | Negative |
| 14 | Positive | Positive | Positive | Positive | Positive |
| | | | | | (100 ng/ml) |
| 15 | Negative | Negative | Negative | Negative | Negative |
| 16 | Negative | Negative | Negative | Negative | Negative |
| 17 | Negative | Negative | Positive | Positive | Positive |
| | | | | | (35 ng/ml) |
| 18 | Negative | Negative | Negative | Negative | Negative |
| 19 | Negative | Negative | Positive | Positive | Positive |
| | | | | | (25 ng/ml) |
| 20 | Negative | Negative | Negative | Negative | Negative |

TABLE 2

| Sample | Interfering Substance | | | |
|---|---|---|---|---|
| # | Measured by Dipstix-7 | LDA | RWB | PCR |
| 1 | 1 + Leukocytes | Negative | Negative | |
| 2 | 1 + Leukocytes | Negative | Negative | |
| 3 | 1 + Leukocytes | Negative | Negative | |
| 4 | 1 + Leukocytes | Negative | Negative | |
| 5 | 1 + Leukocytes | Negative | Negative | |
| 6 | 1 + Leukocytes | Negative | Negative | |
| 7 | 1 + Leukocytes | Negative | Negative | |
| 8 | 2 + Leukocytes | Negative | Negative | |
| 9 | 2 + Leukocytes | Negative | Negative | |
| 10 | 3 + Leukocytes | Negative | Negative | |
| 11 | 3 + Leukocytes | Negative | Negative | |
| 12 | 1 + Leukocytes + Nitrites | + | Negative | Negative |
| 13 | 2 + Leucocytes + Nitrites | ++ | + | Negative |
| 14 | 2 + Leucocytes + Nitrites | +++ | Negative | Negative |
| 15 | 2 + Leucocytes + Nitrites | +++ | + | Negative |
| 16 | 3 + Leucocytes + Nitrites | +++ | + | Negative |
| 17 | Nitrite + | + | + | Negative |
| 18 | Nitrite + | +++ | Negative | Negative |
| 19 | Nitrite + | + | Negative | Negative |
| 20 | Nitrite + | +++ | + | Negative |
| 21 | 3 + Red Blood Cells | Negative | Negative | |
| 22 | 3 + Red Blood Cells | Negative | Negative | |
| 23 | 3 + Red Blood Cells | Negative | Negative | |
| 24 | 3 + Red Blood Cells | Negative | Negative | |
| 25 | 3 + Red Blood Cells | Negative | Negative | |
| 26 | 3 + Red Blood Cells | Negative | Negative | |
| 27 | 3 + Red Blood Cells | + | Negative | |
| 28 | 3 + Red Blood Cells | + | Negative | |
| 29 | 3 + Red Blood Cells | Negative | Negative | |

TABLE 3

| Sample | | |
|---|---|---|
| # | CSF Sample Type | LDA Result |
| | Positive Control (50 ng/ml) | Positive |
| | Positive Control (25 ng/ml) | Positive |
| | Positive Control (12.5 ng/ml) | Positive |
| | Positive Control (6 ng/ml) | Positive |
| | Positive Control (3 ng/ml) | Negative |
| | Negative Control (0 ng/ml) | Negative |
| | High Positive | Positive |
| | Low Positive | Positive |
| | Low Positive | Positive |
| | Negative Control | Negative |
| 1 | Control CSF | Negative |
| 2 | Control CSF | Negative |
| 3 | Control CSF | Negative |
| 4 | Control CSF | Negative |
| 5 | Control CSF | Negative |
| 6 | Control CSF | Negative |
| 7 | Control CSF | Negative |
| 8 | Control CSF | Negative |
| 9 | Control CSF | Negative |
| 10 | Control CSF | Negative |
| 11 | 50 ng/ml Spiked CSF | Positive |
| 12 | 50 ng/ml Spiked CSF | Positive |
| 13 | 50 ng/ml Spiked CSF | Positive |
| 14 | 25 ng/ml Spiked CSF | Positive |
| 15 | 25 ng/ml Spiked CSF | Positive |
| 16 | 25 ng/ml Spiked CSF | Positive |
| 17 | 20 ng/ml Spiked CSF | Positive |
| 18 | 20 ng/ml Spiked CSF | Positive |
| 19 | 20 ng/ml Spiked CSF | Positive |
| 20 | 15 ng/ml Spiked CSF | Positive |
| 21 | 15 ng/ml Spiked CSF | Positive |
| 22 | 15 ng/ml Spiked CSF | Positive |
| 23 | Blood Spiked CSF | Negative |
| 24 | Blood Spiked CSF | Negative |
| 25 | Blood Spiked CSF | Negative |
| 26 | Blood Spiked CSF | Negative |
| 27 | Blood Spiked CSF | Negative |
| 28 | Blood Spiked CSF | Negative |
| 29 | Blood Spiked CSF | Negative |
| 30 | Blood Spiked CSF | Negative |
| 31 | Blood Spiked CSF | Negative |
| 32 | Blood Spiked CSF | Negative |
| 33 | Blood Spiked CSF | Negative |
| 34 | Blood Spiked CSF | Negative |

1. A method for detecting a target antigen in a fluid sample of an individual, the method comprising:
a) providing the fluid sample containing the target antigen;
b) optionally concentrating the fluid sample;
c) passing the fluid sample through a membrane suitable for binding the target antigen, thereby binding the target antigen to the membrane; and
d) detecting the target antigen bound to the membrane of step c) using an antibody specific to the target antigen.

2. The method of claim 1 wherein the optional concentration of step b) comprises centrifuging the fluid sample, thereby forming a target antigen-containing pellet that is resuspended or denatured in solution prior to passing through the membrane of step c).

3. The method of claim 1 wherein the optional concentration of step b) comprises evaporating an appropriate volume of liquid from the fluid sample, thereby concentrating the target antigen in the fluid sample.

4. The method of claim 1 wherein the optional concentration of step b) comprises precipitating the target antigen, thereby forming a target antigen precipitate that is resuspended in solution prior to passing through the membrane of step c).

5. The method of claim 1 wherein detecting the target antigen of step d) comprises a direct detection method.

6. The method of claim 5 wherein the direct detection method further comprises a chromogenic assay.

7. The method of claim 6 wherein the chromogenic assay includes the use of reagents selected from the group consisting of horseradish peroxidase, alkaline phosphatase, and β-galactosidase.

8. The method of claim 1 wherein detecting the target antigen of step d) comprises an indirect detection method.

9. The method of claim 8 wherein the indirect detection method further comprises a chromogenic assay.

10. The method of claim 9 wherein the chromogenic assay includes the use of reagents selected from the group consisting of horseradish peroxidase, alkaline phosphatase, and β-galactosidase.

11. The method of claim 1 wherein the antibody specific to the target antigen is selected from the group consisting of monoclonal antibody and polyclonal antibody.

12. The method of claim 1 wherein the fluid sample is urine.

13. The method of claim 1 wherein the fluid sample is cerebral spinal fluid.

14. The method of claim 1 wherein the fluid sample is synovial fluid.

15. The method of claim 1 wherein the target antigen is expressed by a bacteria.

16. The method of claim 1 wherein the target antigen is expressed by a pathogen selected from the group consisting of parasite, fungus, mold, and virus.

17. The method of claim 1 wherein the target antigen is a protein.

18. The method of claim 15 wherein the bacteria is B. burgdorferi.

19. The method of claim 15 wherein the bacteria is the causative agent of Lyme disease.

20. The method of claim 18 wherein the antibody specific to the target antigen is polyclonal rabbit anti-B. burgdorferi antibody selected from the group consisting of anti-18 kD, anti-23-25 kD, anti-30 kD, anti-31 kD, anti34 kD, anti-39 kD, anti-45 kD, anti-58 kD, anti-66 kD, and anti-83/93 kD.

21. The method of claim 1 wherein the individual is a mammal.

22. The method of claim 21 wherein the mammal is human.

23. The method of claim 1 wherein passing the fluid sample through a membrane of step c) comprises using a microfiltration system.

24. The method of claim 1 wherein passing the fluid sample through a membrane of step c) comprises using gravity.

25. The method of claim 1 wherein the membrane is selected from the group consisting of nitrocellulose, activated paper, and activated nylon membrane.
